(19) United States
(12) Patent Application Publication
(10) Pub. No.: US 2023/0170088 A1
(43) Pub. Date: Jun. 1, 2023

(54) MEDICAL INFORMATION PROCESSOR, MEDICAL INFORMATION PROCESSING METHOD, AND OPERATING ROOM NETWORK SYSTEM

(71) Applicant: SONY GROUP CORPORATION, TOKYO (JP)

(72) Inventors: YUKI SUGIE, Kanagawa (JP); KOJI KASHIMA, Kanagawa (JP); KENTA YAMAGUCHI, Kanagawa (JP)

(21) Appl. No.: 18/159,301

(22) Filed: Jan. 25, 2023

Related U.S. Application Data
(63) Continuation of application No. 16/977,990, filed on Sep. 3, 2020.

(30) Foreign Application Priority Data
Mar. 12, 2018 (JP) 2018-044637

(51) Int. Cl. G16H 40/67; G16H 20/40; G16H 40/20; G16H 15/00; G16H 50/70; G16H 50/20; G16H 40/40; G16H 40/63; G16H 70/20; A61B 17/00; G06V 10/75

(52) U.S. Cl. CPC G16H 40/67; G16H 20/40; G16H 40/20; G16H 15/00; G16H 50/70; G16H 50/20; G16H 40/40; G16H 40/63; G16H 70/20; A61B 17/00; G06V 10/751; A61B 5/02042

(57) ABSTRACT

To make it possible to fully utilize output data from a plurality of apparatuses used during surgery. There is provided a medical information processor including: an acquisition unit that acquires output data of a plurality of apparatuses coupled to an operating room network; a first analysis unit that performs an analysis on a basis of a correlation among the output data; and an output control section that controls an output of a result of the analysis.

### CROSS REFERENCE TO RELATED APPLICATIONS

This application is a continuation application of U.S. patent application Ser. No. 16/977,990, filed on Sep. 3, 2020, is a U.S. National Phase of International Patent Application No. PCT/JP2019/004535 filed on Feb. 8, 2019, which claims priority benefit of Japanese Patent Application No. JP 2018-044637 filed in the Japan Patent Office on Mar. 12, 2018. Each of the above-referenced applications is hereby incorporated herein by reference in its entirety.

### TECHNICAL FIELD

The present disclosure relates to a medical information processor, a medical information processing method, and an operating room network system.

### BACKGROUND ART

Improving surgical efficiency or surgical achievement is of great interest in the medical industry, and apparatuses, methods, or systems have been actively developed to improve the surgical efficiency or surgical achievement. For example, PTL 1 listed below discloses a technique of analyzing behaviors of an operator or an assistant and reducing medical accidents to thereby improve the surgical efficiency or surgical achievement.

### CITATION LIST

### Patent Literature

PTL 1: Japanese Unexamined Patent Application Publication No. 2004-157614

### SUMMARY OF THE INVENTION

### Problem to be Solved by the Invention

In existing technologies including the technique of PTL 1, however, output data from a plurality of apparatuses used during surgery have been only utilized for the analysis based on the behaviors of the operator or the assistant in many cases, and have not been able to be fully utilized.

In view of the above issues, it is an object of the present disclosure to provide a medical information processor, a medical information processing method, and an operating room network system which are novel and improved and make it possible to fully utilize output data from a plurality of apparatuses used during surgery.

### Means for Solving the Problem

According to the present disclosure, there is provided a medical information processor including: an acquisition unit that acquires output data of a plurality of apparatuses coupled to an operating room network; a first analysis unit that performs an analysis on a basis of a correlation among the output data; and an output control section that controls an output of a result of the analysis.

In addition, according to the present disclosure, there is provided a medical information processing method implemented by a computer, in which the method includes: acquiring output data of a plurality of apparatuses coupled to an operating room network; performing an analysis on a basis of a correlation among the output data; and controlling an output of a result of the analysis.

In addition, according to the present disclosure, there is provided an operating room network system including: a plurality of apparatuses coupled to an operating room network; and a medical information processor that analyzes output data of the plurality of apparatuses, in which the medical information processor includes an acquisition unit that acquires the output data, a first analysis unit that performs an analysis on a basis of a correlation among the output data, and an output control section that controls an output of a result of the analysis.

### Effect of the Invention

As described above, according to the present disclosure, it is possible to fully utilize output data from a plurality of apparatuses used during surgery.

It is to be noted that the above-mentioned effects are not necessarily limitative; in addition to or in place of the above effects, there may be achieved any of the effects described in the present specification or other effects that may be grasped from the present specification.

### BRIEF DESCRIPTION OF THE DRAWINGS

FIG. 1 is a block diagram illustrating a configuration example of an operating room network system.

FIG. 2 describes an example of processing of a medical information processor 100.

FIG. 3 is a block diagram of a functional configuration example of the medical information processor 100.

FIGS. 4A and 4B describe the specific examples related to determination of presence or absence of occurrence of an event of bleeding performed by an event determination section 112.

FIG. 5 illustrates an example of a table generated by an event zone analysis section 113.

FIG. 6 describes an example of analysis processing performed by the event zone analysis section 113.

FIG. 7 describes an example of an output control performed by an output control section 114.

FIG. 8 is a flowchart illustrating an example of a flow of processing performed by the medical information processor 100.

FIG. 9 is a flowchart illustrating an example of a flow of processing performed by the medical information processor 100.

FIGS. 10A and 10B describe an examples of processing of changing a sampling interval of data.

FIGS. 11A and 11B describe an examples of processing of analyzing an indication of occurrence of an event.

FIG. 12 describes a second embodiment according to the present disclosure.

FIG. 13 is a block diagram illustrating a hardware configuration example of the medical information processor 100.

### MODES FOR CARRYING OUT THE INVENTION

Hereinafter, description is given in detail of preferred embodiments of the present disclosure with reference to the accompanying drawings. It is to be noted that, in the present specification and drawings, repeated description is omitted for components substantially having the same functional configuration by assigning the same reference numerals.

It is to be noted that description is given in the following order.

### 1. First Example

1.1. Overview

1.2. Systems Configuration Example

1.3. Functional Configuration Example

1.4. Processing Flow

1.5. Processing Variation

### 2. Second Embodiment

### 3. Third Embodiment

### 4. Fourth Embodiment

### 5. Other Embodiments

### 6. Hardware Configuration Example

### 7. Conclusion

### 1. FIRST EMBODIMENT

### 1.1. Overview

First, description is given of an overview of a first embodiment according to the present disclosure.

As described above, improving surgical efficiency or surgical achievement is of great interest in the medical industry, and apparatuses, methods, or systems have been actively developed to improve the surgical efficiency or the surgical achievement.

For example, standardization of surgical procedures performed at various academic societies has achieved improvement and more efficiency in surgeries for various diseases and various illnesses. However, the standardization requires a considerable amount of time to accumulate know-hows of the surgical procedures, and there is also a limitation on the range of standardization (e.g., arrangement of apparatuses, image quality setting of a monitor or an endoscopic system, and the like fall outside the standardization in many cases).

In order to solve this, an apparatus, a method, or a system has been developed, which automatically collects data regarding surgery and analyzes the data to thereby promote an improvement in the surgery. One of those is the technique disclosed in PTL 1 listed above. PTL 1 discloses a technique of analyzing behaviors of an operator or an assistant and reducing medical accidents to thereby improve the surgical efficiency or the surgical achievement.

In existing technologies including the technique of PTL 1, however, output data from a plurality of apparatuses used during surgery have been only utilized for the analysis based on the behaviors of the operator or the assistant in many cases, and have not been able to be fully utilized.

In addition, the technique of PTL 1 prescribes an apparatus to be measured and information to be measured in advance; it is assumed that the technique is used in a state in which an analysis method to be performed using the information is also prescribed. That is, the technique of PTL 1 aims to provide information useful to a user from information known to have a correlation in advance, and is not able to provide information useful to the user by extracting a correlation from information of which presence or absence of the correlation is unclear.

In addition, the technique of PTL 1 is not able to output information regarding a method for improving a surgical achievement (hereinafter, for the sake of convenience, “outputting information regarding the method for improving the surgical achievement” or “information itself regarding the method for improving the surgical achievement” may be referred to as a “feedback” in some cases) by analyzing information acquired during surgery. Further, the technique of PTL 1 is not able to perform a feedback in a case where a new apparatus (e.g., a product of another company or a new product, etc.), of which specifications (e.g., data unit, data format, data type (e.g., data dimension, etc.), delay information, apparatus type, or various parameters, etc. (e.g., resolution or image quality parameters, etc.) are unclear, is used for surgery.

In view of the above circumstances, the discloser of the present application has devised a technique according to the present disclosure. The medical information processor 100 according to the present disclosure is able to perform analysis on the basis of a correlation among output data of a plurality of apparatuses coupled to an operating room network and to perform various feedbacks to an operator or an assistant using results of the analysis.

Hereinafter, description is given in detail of the present disclosure. It is to be noted that, in the following, description is given of a case where the present disclosure is applied to the medical information processor 100, but there is no limitation on an object to which the present disclosure is applied. For example, the present disclosure may be applied to a medical information processing method or an operating room network system, etc.

### 1.2. System Configuration Example

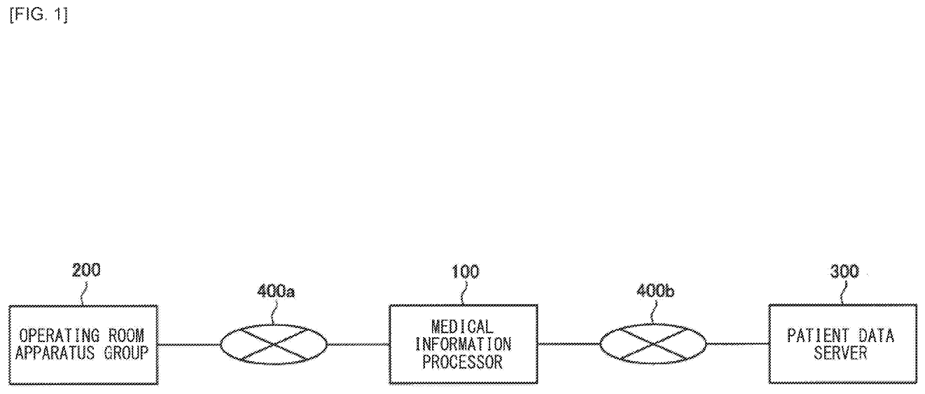

In the above, description has been given of the overview of the first embodiment according to the present disclosure. Next, description is given, with reference to FIG. 1, of a configuration example of an operating room network system according to the present embodiment.

As illustrated in FIG. 1, the operating room network system according to the present embodiment includes the medical information processor 100, an operating room apparatus group 200, and a patient data server 300. In addition, the medical information processor 100 and the operating room apparatus group 200 are coupled to each other by a network 400a, and the medical information processor 100 and the patient data server 300 are coupled to each other by a network 400b.

(Medical Information Processor 100)

The medical information processor 100 is an apparatus that analyzes output data of a plurality of apparatuses (operating room apparatus group 200) coupled to the operating room network. More specifically, the medical information processor 100 acquires output data of the operating room apparatus group 200 via the network 400a, and performs an analysis on the basis of the correlation among the output data. In addition, the medical information processor 100 is able to provide various feedbacks to the operator or the assistant using results of the analysis.

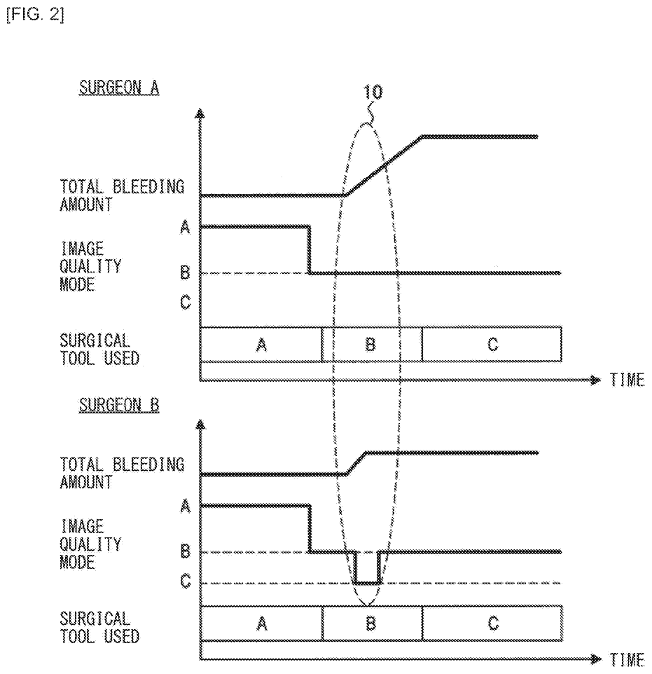

Description is given now, with reference to FIG. 2, of an example of processing of the medical information processor 100. For example, the medical information processor 100 acquires and analyzes the output data of the operating room apparatus group 200 to thereby recognize that a patient under surgery has bled. Then, the medical information processor 100 analyzes various types of output data at the time when bleeding occurs in surgeries of the same operative method (or similar operative method) performed in the past to thereby search for information correlated with a bleeding amount.

For example, FIG. 2 illustrates chronological changes in a total bleeding amount, image quality modes of a monitor, and surgical tools used for each of a surgeon A and a surgeon B in a case where the surgeon A and the surgeon B each perform surgery of the same operative method. The medical information processor 100 analyzes the information, and recognizes that there is a correlation between the total bleeding amount and the image quality modes of the monitor, as illustrated in a red frame 10. More specifically, it is suggested that, in a case where the surgeon B changes the image quality mode of the monitor from B to C immediately after bleeding, the total bleeding amount is suppressed more than a case where the image quality mode of the monitor remains B without being changed even after the bleeding as in the surgeon A. The medical information processor 100 recognizes this correlation to thereby be able to perform a feedback proposing to change the image quality mode of the monitor to C as a countermeasure to the case where bleeding occurs.

It is to be noted that, in the example of FIG. 2, the medical information processor 100 extracts correlations only from two examples; however, the medical information processor 100 is able to provide a more highly reliable feedback if similar correlations are extracted from more cases. In addition, in a case where a correlation is able to be extracted, the medical information processor 100 may determine a degree of an influence of the correlation exerted on a surgical achievement to thereby make a more effective proposal. For example, the medical information processor 100 may use, as data (index) evaluating the surgical achievement, data regarding a bleeding amount, data regarding surgery time, data regarding a hospitalization period, or data regarding a survival rate (e.g., five-year survival rate, etc.), data indicating an incidence rate of pathology caused by surgery, such as complication, to preferentially perform a feedback regarding output data having a greater correlation with the data. It is to be noted that data regarding a bleeding amount is a concept including, not only the bleeding amount itself, but also various factors related to the bleeding amount (e.g., factors that increase or decrease the bleeding amount); the same also applies to other data, etc. regarding the surgery time.

Here, as described with reference to FIG. 2, the medical information processor 100 extracts a correlation from among the acquired output data, and does not perform correlation analysis on the information in a state in which the correlation has been found to be present in advance between the total bleeding amount and the image quality mode of the monitor. Accordingly, the medical information processor 100 according to the present disclosure distinctly differs from the apparatus disclosed in PTL 1 listed above.

Further, the medical information processor 100 is able to analyze output data acquired during surgery and to perform the feedback as described above during the surgery. It has been common to make efforts to review a surgical procedure after surgery to thereby improve subsequent surgical procedures. However, the application of the present disclosure allows the above-described feedback to be performed during the surgery, thereby enabling the operator to improve the surgical procedure in real time and thus to reduce a surgical failure. It is to be noted that those described above are merely exemplary, and the medical information processor 100 may analyze data acquired during time outside the surgery (e.g., before or after the surgery) and may perform a feedback during the time outside the surgery (e.g., before or after the surgery). Besides, the medical information processor 100 is able to create various effects. Description is given later of details of the medical information processor 100.

(Operating Room Apparatus Group 200)

The operating room apparatus group 200 is a collection of a plurality of apparatuses installed in an operating room and used for a surgery. For example, the operating room apparatus group 200 includes an endoscopic system, a wearable device (e.g., wearable device, etc. worn by an operator or patient), a blood tank, a shadowless lamp, a monitor, an operating table, or a surgical field camera, etc. It is to be noted that the apparatus included in the operating room apparatus group 200 is not limited thereto, and any apparatus may be used as long as the apparatus is an apparatus to be used in a surgery (or an apparatus related to a surgery). In addition, the operating room apparatus group 200 need not necessarily be located in an operating room, but may be located outside the operating room.

(Patient Data Server 300)

The patient data server 300 is an apparatus that manages any information regarding patients. More specifically, the patient data server 300 manages patient attribute information (e.g., name, sex, age, height, body weight, body fat percentage, BMI, blood pressure, vision, hearing, or chronic disease, etc.), information regarding hospitalization (e.g., hospitalization period, hospital room, or personnel in charge, etc.), or various types of historic information (e.g., diagnostic history, treatment history, surgery history, dosing history, or information regarding diagnostic, treatment, surgery or dosing results (e.g., surgery success or failure, surgery time, bleeding amount, or presence or absence of complication, etc.)). It is to be noted that information managed by the patient data server 300 is not limited thereto, and may be any information as long as the information concerns patients.

(Network 400a and Network 400b)

The network 400a and the network 400b are each a wired or wireless transmission path for information communicated by the above-described various apparatuses. For example, the network 400a and the network 400b may each include various types of LAN (Local Area Network) and WAN (Wide Area Network) including Ethernet (registered trademark), and a public network such as the Internet. In addition, the network 400a and the network 400b may each include a private line network such as IP-VPN (Internet Protocol-Virtual Private Network) or a short-range wireless communication network such as Bluetooth (registered trademark). It is to be noted that the network 400a and the network 400b are each provided with various network devices such as a hub, a switch, or a router, as a matter of course, and the number and the specifications thereof are not particularly limited. In addition, in the present embodiment, the network 400a and the network 400b are each also referred to as an “operating room network”.

The description has been given above of the configuration example of the operating room network system according to the present embodiment. It is to be noted that, in the present embodiment, the various apparatuses described above are assumed to be installed in the same hospital (including one of hospitals of which respective locations are coupled by a network). Description is given later of an example of a case where various apparatuses are installed in a plurality of hospitals.

In addition, the configuration described above with reference to FIG. 1 is merely exemplary, and the configuration of the operating room network system according to the present embodiment is not limited to such an example. For example, all or a portion of the functions of the medical information processor 100 may be provided in an external apparatus (including the operating room apparatus group 200 or the patient data server 300). For example, the function of accumulating data from the operating room apparatus group 200 may be implemented in an apparatus different from the medical information processor 100. In addition, the number of the various apparatuses described above is not particularly limited. The configuration of the operating room network system according to the present embodiment may be flexibly modified in accordance with specifications and operations.

### 1.3. Functional Configuration Example

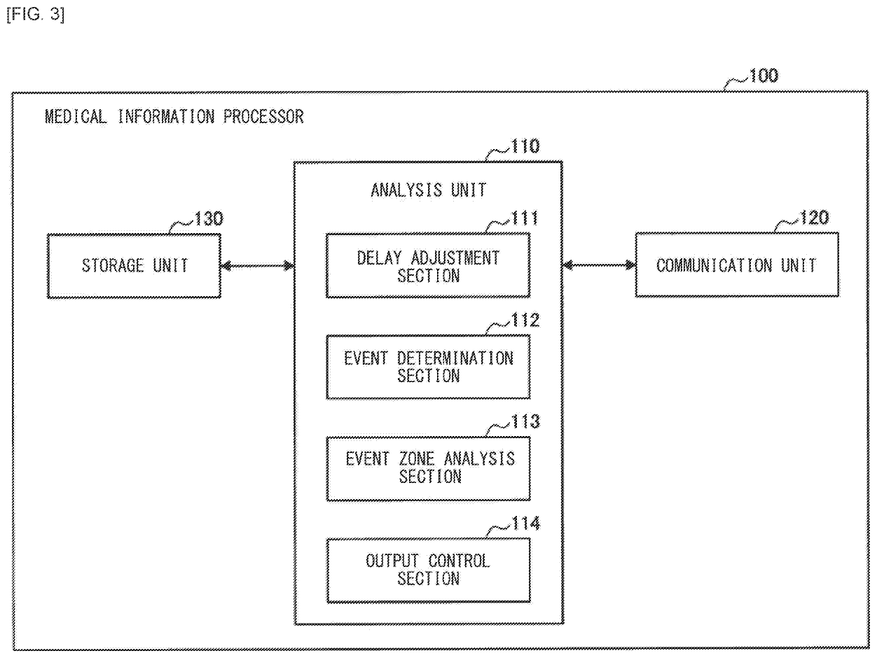

The description has been given above of the configuration example of the operating room network system according to the present embodiment. Subsequently, description is given of a functional configuration example of the medical information processor 100 according to the present embodiment with reference to FIG. 3.

As illustrated in FIG. 3, the medical information processor 100 includes an analysis unit 110, a communication unit 120, and a storage unit 130.

(Analysis Unit 110)

The analysis unit 110 is a functional configuration for functioning as a first analysis unit that performs an analysis on the basis of a correlation among output data from a plurality of apparatuses coupled to the operating room network and that controls an output of a result of the analysis. As illustrated in FIG. 3, the analysis unit 110 includes a delay adjustment section 111, an event determination section 112, an event zone analysis section 113, and an output control section 114. Hereinafter, description is given of each functional configuration included in the analysis unit 110. It is to be noted that, in the following, description is given, as an example, mainly of a case where the analysis unit 110 analyzes output data acquired during surgery to perform a feedback during the surgery; however, as described above, the analysis unit 110 may analyze data acquired during time outside the surgery (e.g., before or after the surgery) to perform a feedback during the time outside the surgery (e.g., before or after the surgery).

(Delay Adjustment Section 111)

The delay adjustment section 111 is a functional configuration for adjusting delays of data from the operating room apparatus group 200. As described above, the operating room apparatus group 200 is a collection of a plurality of apparatuses, and timings at which respective apparatuses output data (in other words, delay amounts at the time when the respective apparatuses output data) are different. Thus, in order for the medical information processor 100 to grasp events that have occurred at the same time, it is required to adjust delays in the output data from the respective apparatuses. Therefore, the delay adjustment section 111 adjusts the delays in the output data of the respective apparatuses.

There is no particular limitation on the method for the delay adjustment section 111 to adjust the delays. For example, in a case where an apparatus included in the operating room apparatus group 200 is able to output a delay amount as meta data, the delay adjustment section 111 may adjust the delays on the basis of the meta data from the respective apparatuses. In addition, in a case where the delay amounts of the respective output data are found on the basis of past achievements or empirical rules, the delay adjustment section 111 may adjust the delays on the basis of manual input by a user, machine learning, or the like.

To describe the adjustment of the delays more specifically, the delay adjustment section 111 advances time corresponding to the output data by a delay amount. For example, in a case where the delay amount is five [ms], the delay adjustment section 111 advances the time corresponding to the output data by five [ms]. The delay adjustment section 111 performs this adjustment on the output data from the respective apparatuses, thereby making it possible to further improve accuracy in correlation extraction of the output data from the respective apparatuses in processing in a subsequent stage. It is to be noted that the method for the delay adjustment section 111 to adjust the delays is not limited to those described above.

(Event Determination Section 112)

The event determination section 112 is a functional configuration for determining presence or absence of occurrence of an event. Description is now given of the “data evaluating a surgical achievement” in the present embodiment, before describing the event and functions of the event determination section 112 in the present embodiment.

The data evaluating the surgical achievement is data included in the output data from the operating room apparatus group 200 (or the patient data server 300), and refers to an index evaluating the surgical achievement. For example, the data evaluating the surgical achievement include data regarding a bleeding amount, data regarding surgery time, data regarding a hospitalization period, or data regarding a survival rate (e.g., five-year survival rate, etc), etc. It is to be noted that, as described above, the data regarding the bleeding amount is a concept including, not only the bleeding amount itself, but also various factors related to the bleeding amount (e.g., factors that increase or decrease the bleeding amount); the same also applies to other data, etc. regarding the surgery time. In addition, the data evaluating the surgical achievement is not limited to the index.

The event in the present embodiment refers to an event that influences the data evaluating the surgical achievement. For example, in a case where the data evaluating the surgical achievement is a “bleeding amount,” the event may be an event of “bleeding (e.g., bleeding in which a bleeding variation exceeds a predetermined threshold value, etc.) that influences the bleeding amount. In addition, in a case where the data evaluating the surgical achievement is “surgery time”, the event may be an event of “smoke generation (e.g., smoke generation, etc. in which a variation in smoke generated by use of an energy device such as an electric scalpel exceeds a predetermined threshold value)” that influences the surgery time. It is to be noted that those described above are merely examples of the event, and the content of the event is not limited thereto.

In addition, the event determination section 112 determines presence or absence of occurrence of the event as described above by various methods. More specifically, the event determination section 112 analyzes an image captured by the surgical field camera to thereby be able to determine presence or absence of occurrence of an event. For example, the event determination section 112 compares a feature amount upon bleeding extracted from captured images acquired in the past surgeries and a feature amount of a captured image acquired in an ongoing surgery with each other, to thereby be able to predict a bleeding variation in the ongoing surgery and to determine that an event of bleeding has occurred in a case where the bleeding variation exceeds a predetermined threshold value (hereinafter, may be referred to as a “bleeding variation threshold value” in some instances). It is to be noted that the bleeding variation refers to a variation in a total bleeding amount per unit time.

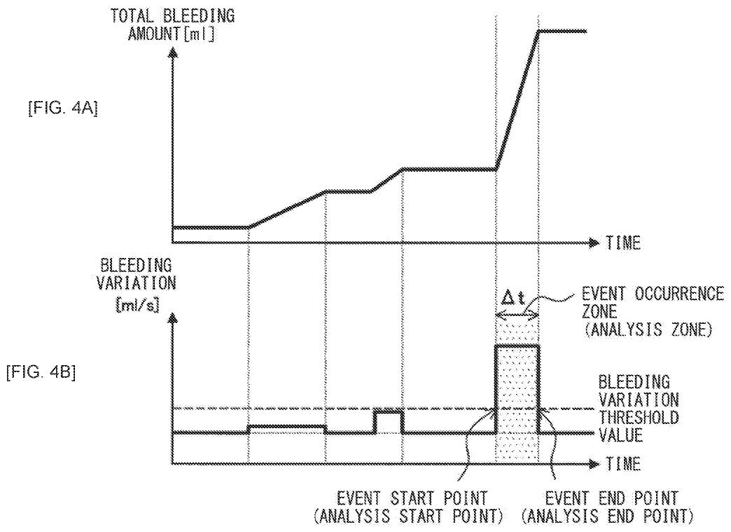

Description is now given, with reference to FIGS. 4A and 4B, of a specific example related to the determination of presence or absence of occurrence of the event of bleeding to be performed by the event determination section 112. FIG. 4A illustrates a total bleeding amount at each time, and FIG. 4B illustrates a bleeding variation at each time. As illustrated in FIG. 4B, the event determination section 112 determines that a zone (period) in which the bleeding variation is equal to or greater than the bleeding variation threshold value is a zone in which an event has occurred (referred to as an “event occurrence zone” in the drawing).

In addition, the event determination section 112 is able to determine presence or absence of occurrence of an event not only in an ongoing surgery but also in surgeries performed in the past. More specifically, the event determination section 112 analyzes various data acquired from the operating room apparatus group 200 in the past surgeries and stored in the storage unit 130 to thereby be able to determine presence or absence of occurrence of an event and to output a zone in which the event has occurred. It is to be noted that the determination method of presence or absence of occurrence of an event in surgeries performed in the past is similar to those described above. In addition, as for the surgeries performed in the past, the event determination section 112 may determine in advance presence or absence of occurrence of an event to store a determination result in the storage unit 130.

It is to be noted that the determination method of presence or absence of occurrence of an event performed by the event determination section 112 is not limited thereto; as long as the data acquired from the operating room apparatus group 200 are used, the event determination section 112 is able to determine presence or absence of occurrence of an event using an arbitrary method. For example, in a case where an amount of blood accumulated in the blood tank is measurable, the event determination section 112 may recognize a speed at which the blood amount (bleeding amount) increases, a total amount thereof, and the like by communicating with the blood tank to determine presence or absence of occurrence of an event on the basis of the information.

In addition, the event determination section 112 is able to appropriately change the threshold value used in the processing of determining presence or absence of occurrence of an event (in the above-described example, the bleeding variation threshold value). More specifically, in a case where an analysis result outputted by the event zone analysis section 113 in processing in a subsequent stage is not statistically significant, the event determination section 112 appropriately changes the threshold values used in the processing of determining presence or absence of occurrence of an event. This allows a zone in which an event occurs to be changed, thus allowing the event zone analysis section 113 to have a higher possibility of being able to output statistically significant analysis results.

In a case where it is determined that an event has occurred, the event determination section 112 notifies the event zone analysis section 113 of information regarding an event occurrence zone (e.g., information regarding a time point of occurrence and a time point of ending of the event, etc.) in the ongoing surgery and the past surgeries.

(Event Zone Analysis Section 113)

The event zone analysis section 113 is a functional configuration for performing an analysis on the basis of a correlation among data in a zone in which an event has occurred. More specifically, the event zone analysis section 113 uses information regarding the event occurrence zone notified from the event determination section 112 to record data of the operating room apparatus group 200 in the event occurrence zone. More specifically, as for the ongoing surgery, the event zone analysis section 113 records various data acquired from the operating room apparatus group 200 in the event occurrence zone. In addition, as for the past surgeries, the event zone analysis section 113 acquires, from the storage unit 130, various data acquired from the operating room apparatus group 200 in the event occurrence zone. Further, the event zone analysis section 113 acquires, from the patient data server 300, information regarding patients who have undergone surgeries for both of the ongoing surgery and the past surgeries.

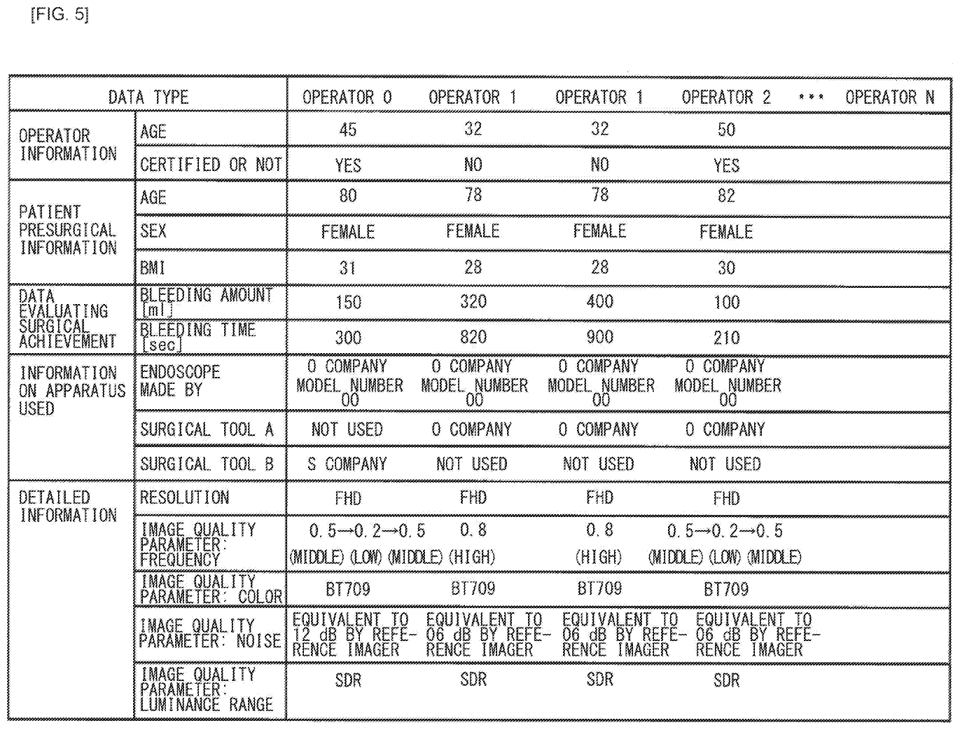

This allows the event zone analysis section 113 to generate a table as illustrated in FIG. 5. More specifically, as illustrated in FIG. 5, the event zone analysis section 113 generates a table including operator information, patient pre-surgery information, data evaluating a surgical achievement, information on an apparatus used, and detailed information, etc. The table of FIG. 5 includes, in a case where the data evaluating the surgical achievement is “bleeding amount”, data of past surgeries in which “bleeding” occurred as in the case of the ongoing surgery (the reason why two records of the data of an operator 1 are included is because bleeding occurred twice in the same surgery).

It is assumed, but not limited to, that the operator information, the data evaluating a surgical achievement, the information on an apparatus used, and the detail information are acquired from the operating room apparatus group 200 or the storage unit 130, and that the patient pre-surgery information is acquired from the patient data server 300. It is to be noted that, instead of performing the above-described processing on all of the past surgeries, the event zone analysis section 113 may perform the above-described processing on surgeries of which patients, pathologies (degree of symptoms, etc.), operators, or surgery contents are highly similar. This enables the event zone analysis section 113 to improve analysis accuracy and thus to reduce a load of analysis processing.

In addition, the event zone analysis section 113 calculates a correlation between the data evaluating the surgical achievement and other output data (including output data of a plurality of apparatuses linked to the operating room network) to thereby extract factors influencing the data evaluating the surgical achievement. For example, the event zone analysis section 113 performs multiple regression analysis according to the following (Expression 1) on the basis of each of data illustrated in the table of FIG. 5. More specifically, when the data evaluating the surgical achievement (e.g., bleeding amount) is set as an objective function y and a value indicating other output data is set as an explanatory variable x, the following (Expression 1) holds true. Here, in (Expression 1), a is a coefficient of each explanatory variable, p is a number of a factor, and c is a residual. The event zone analysis section 113 performs an analysis of variance (e.g., F-test, etc.) which assumes null hypothesis that a multiple correlation coefficient in the population for a regression line obtained by multiple regression analysis is zero, and performs a test (e.g., t-test, etc.) which assumes null hypothesis that a partial regression coefficient is not zero, to thereby extract a factor that influences the data evaluating the surgical achievement.

$$y = {a}_{0} + {\sum }_{j = 1}^{p} {a}_{j} \cdot {x}_{j} + ε \qquad ( Expression 1 )$$

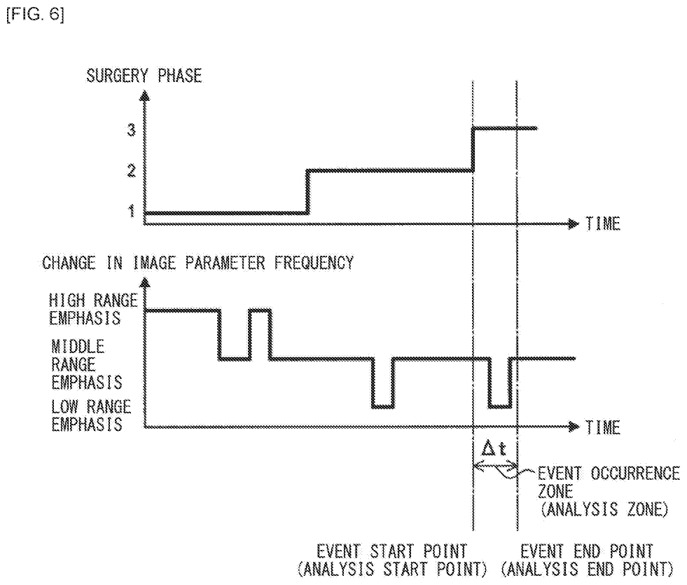

For example, as illustrated in FIG. 6 (or data of an operator 0 and data of an operator 2 in FIG. 5), it is assumed that a frequency of the image quality parameter is changed from “middle range emphasis” to “low range emphasis” in the event occurrence zone in the past surgery and is further returned from the “low range emphasis” to the “middle range emphasis”, and that thereafter the event “bleeding” ends. In a case where the data other than the frequency of the image quality parameter has not changed largely, the event zone analysis section 113 outputs the frequency of the image quality parameter as a factor that contributes the most to the data evaluating the surgical achievement (bleeding amount in this example).

In a case where a statistically significant factor is extracted by the multiple regression analysis described above, the event zone analysis section 113 ends the factor extraction processing. Meanwhile, in a case where no statistically significant factor is extracted, the event determination section 112, as described above, changes the event occurrence zone by appropriately changing the threshold value to be used in the processing of determining presence or absence of occurrence of an event, and the event zone analysis section 113 repeats a series of processing of performing the multiple regression analysis again on data of the changed zone a predetermined number of times. It is to be noted that there may be a plurality of factors extracted by the event zone analysis section 113 in the above-described processing. In addition, the method for the event zone analysis section 113 to analyze the correlation between the data evaluating the surgical achievement and other output data is not limited to the multiple regression analysis, as long as the method is able to analyze the correlation. For example, the method for the event zone analysis section 113 to analyze the correlation may be principal component analysis, cluster analysis, or an analysis by machine learning, etc.

Here, in the analysis by machine learning in the event zone analysis section 113, for example, a neural network is used to generate a classifier or an estimator, that is learned by learning data in which data evaluating a surgical achievement and output data of a plurality of apparatuses linked to an operating room network are associated with each other, and the output data of the plurality of apparatuses linked to the operating room network during surgery are inputted to the classifier or the estimator, thereby making it possible to predict and output a future surgical achievement. In addition, similar surgeries in the past with better surgical achievements than the expected surgical achievement may be calculated, and differences in output values among the plurality of apparatuses in the surgeries may be statistically or regressively analyzed, to output a method for improving the surgical achievement on the basis of results of the analysis.

In addition, on the basis of the factor (statistically significant factor) that have contributed significantly to the data evaluating the surgical achievement (bleeding amount in this example), the event zone analysis section 113 is able to output the method for improving the surgical achievement. For example, in a case where the factor that has contributed significantly to the data evaluating the surgical achievement is the frequency of the image quality parameter, the event zone analysis section 113 applies the frequency of the image quality parameter, which may be determined to be optimal on the basis of data acquired during the past surgeries, also to the ongoing surgery. There is no particular limitation on how to derive the method for improving the surgical achievement (e.g., an optimal set value, etc. of the frequency of the image quality parameter). For example, the event zone analysis section 113 may employ the same method as that of the past similar surgery with the best surgical achievement (e.g., a set value, etc. of the past similar surgery with the least bleeding amount).

The event zone analysis section 113 provides, to the output control section 114, information regarding the method for improving the surgical achievement. It is to be noted that the event zone analysis section 113 may calculate a degree of recommendation (or reliability) on the basis of analysis results (such as high statistical significance), and may provide, to the output control section 114, the information regarding the method for improving the surgical achievement with information regarding such a degree of recommendation being included.

Here, in a case where there are sufficiently many surgical achievements to be fed back, an adequate surgical procedure has often already been determined (in other words, the surgical procedure has already been standardized), thus causing the degree of recommendation (or reliability) of the analysis results to be relatively lower than existing surgical procedures. Meanwhile, as there are fewer surgical achievements to be fed back, the adequate surgical procedure has not been determined (in other words, the surgical procedure has not been standardized) in more cases, thus causing the degree of recommendation (or reliability) of the analysis results to be relatively higher than the existing surgical procedures. From those described above, the event zone analysis section 113 may calculate the degree of recommendation (or reliability) on the basis of the surgical achievement to be fed back, or the like. It is to be noted that the content of the processing by the event zone analysis section 113 is not limited to those described above.

(Output Control Section 114)

The output control section 114 is a functional configuration for controlling output of information regarding the method for improving the surgical achievement (in other words, controlling the feedback). More specifically, the output control section 114 generates control information for controlling an external apparatus (e.g., apparatuses, etc. included in the operating room apparatus group 200) on the basis of information regarding the method for improving the surgical achievement provided from the event zone analysis section 113, and provides the control information to the external apparatus, to thereby control the feedback. For example, the output control section 114 may provide control information to a monitor included in the operating room apparatus group 200 during surgery to thereby cause the monitor to display the feedback.

Here, description is given of a “phase” in the present embodiment, in explaining the control of the feedback by the output control section 114. The surgery involves standard procedures, and a proceeding (or recommended proceeding) has been determined in many cases. The “phase” in the present embodiment refers to a segment in this proceeding. The output control section 114 recognizes a phase in the surgery by analyzing various data provided from the operating room apparatus group 200. For example, the output control section 114 analyzes a captured image provided from a surgery field camera to thereby be able to recognize the phase in the surgery. It is to be noted that the output control section 114 may recognize the phase in the surgery by manual entry by the user. For example, the user may perform a predetermined input (e.g., pressing predetermined buttons) at a timing when the phase changes to thereby cause the output control section 114 to recognize the phase in the surgery.

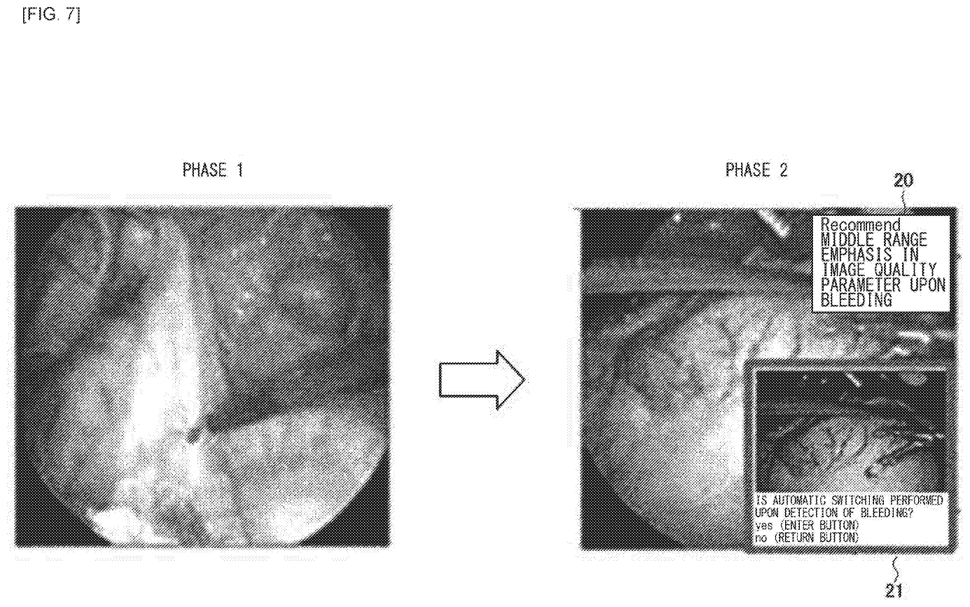

Then, the output control section 114 recognizes the phase in the surgery, and causes the external apparatus to output the feedback at an adequate phase (or adequate timing). For example, obtainment of an analysis result that “in a case where bleeding occurs in a phase 2, it is recommended to set the frequency of the image quality parameter to the middle range emphasis” allows the output control section 114 to change the phase of the surgery from a “phase 1” to the “phase 2” as illustrated in FIG. 7 and to cause the external apparatus to output a feedback 20 at a timing when bleeding occurs (i.e., no feedback is outputted even when the bleeding occurs in the phase 1). This enables an operator to confirm the feedback at an adequate timing.

In addition, as illustrated in the feedback 20 of FIG. 7, the output control section 114 may indicate that the feedback 20 is merely a matter of recommendation. More specifically, a character string “Recommend” is displayed in the feedback 20 of FIG. 7 to thereby indicate that the feedback 20 is merely the matter of recommendation. This enables the operator to recognize that the treatment indicated by the feedback is not enforced and thus to determine by himself or herself whether or not to employ the feedback.

In addition, in a case where the information regarding the method for improving the surgical achievement provided by the event zone analysis section 113 includes information regarding the degree of recommendation (or reliability), the output control section 114 may reflect the degree of recommendation in the feedback. More specifically, the output control section 114 may control display contents (e.g., numerical value, graphic, symbol, or character string, etc.), display size, display color (e.g., color of character string, etc., or color of background, etc.), display position, content of sound output, size of sound output, lighting or flashing of a lamp, etc. in the feedback, in accordance with the degree of recommendation. For example, in a case where the degree of recommendation is higher than a predetermined threshold value, the output control section 114 may color the feedback green; in a case where the degree of recommendation is equal to or less than the predetermined threshold value, the output control section 114 may color the feedback red. This enables the operator to intuitively recognize the degree of recommendations of the feedback.

In addition, in a case where the medical information processor 100 has a function of being able to control the operating room apparatus group 200 (e.g., a function of being able to change a setting of an apparatus included in the operating room apparatus group 200, etc.), the output control section 114 may perform a feedback of a guide 21 as to whether or not to control the operating room apparatus group 200. In the guide 21 of FIG. 7, an image (recommended image) after switching is displayed together with character strings of “Is automatic switching performed upon detection of bleeding? Yes (enter button) No (return button)”. This enables the operator to easily select a setting that is easier to view. In a case where the operator activates the automatic switching upon bleeding by pressing the enter button, the medical information processor 100 provides control information to a target apparatus upon detection of bleeding to thereby implement the automatic switching.

It is to be noted that the output control section 114 outputs the guide 21 not to adversely influence the surgery. For example, the output control section 114 outputs the guide 21 in a case where no emergency (e.g., a large amount of bleeding, etc.) has occurred, in a case where a forceps is stationary, or in a case where the movement of a scope is stable, etc. The control content of the feedback by the output control section 114 is not limited to those described above.

(Communication Unit 120)

The communication unit 120 is a functional configuration for functioning as an acquisition unit, and acquires various data by communicating with the operating room apparatus group 200 or the patient data server 300. For example, the communication unit 120 receives various data regarding surgery from the operating room apparatus group 200. In addition, data the communication unit 120 receives various data regarding patients from the patient data server 300. Then, the communication unit 120 transmits control information for controlling the operating room apparatus group 200 (e.g., monitor, etc.) to the operating room apparatus group 200 upon feeding back. It is to be noted that the content and the timing of the communication by the communication unit 120 are not limited thereto.

(Storage Unit 130)

The storage unit 130 is a functional configuration for storing various types of information. For example, the storage unit 130 may store various data acquired from the operating room apparatus group 200 in the past surgeries, a determination result of presence or absence of occurrence of an event, an analysis result of an event occurrence zone, or information regarding the feedback, etc. In addition, the storage unit 130 may store programs or parameters, etc. to be used by each of the functional configurations of the medical information processor 100. It is to be noted that information stored by the storage unit 130 is not limited thereto.

The description has been given above of the functional configuration examples of the medical information processor 100. It is to be noted that the functional configurations described above with reference to FIG. 3 are merely exemplary, and the functional configuration of the medical information processor 100 is not limited to such an example. For example, the medical information processor 100 may not necessarily include all of the functional configurations illustrated in FIG. 3. In addition, the functional configuration of the medical information processor 100 may be flexibly modified in accordance with specifications or operations.

### 1.4. Processing Flow

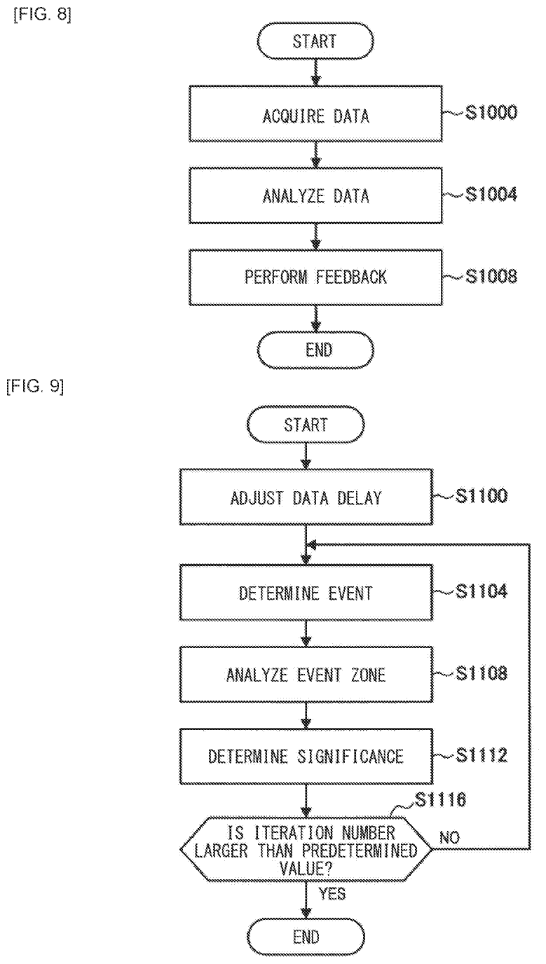

The description has been given above of the functional configuration examples of the medical information processor 100 according to the present embodiment. Subsequently, description is given, with reference to FIGS. 8 and 9, of an example of a flow of processing by the medical information processor 100 according to the present embodiment.

FIG. 8 is a flowchart illustrating an overall flow of the processing performed by the medical information processor 100. In step S1000, the communication unit 120 of the medical information processor 100 communicates with the operating room apparatus group 200 or the patient data server 300 to thereby acquire various data. In step S1004, the analysis unit 110 analyzes the various data. Then, in step S1008, the output control section 114 controls output of information regarding the method for improving the surgical achievement on the basis of results of the analysis by the analysis unit 110 (in other words, controls the feedback).

FIG. 9 is a flow chart illustrating a flow of more detailed processing in step S1004 (processing of analyzing various data by the analysis unit 110) of FIG. 8. In step S1100, the delay adjustment section 111 adjusts a delay in the data acquired from the operating room apparatus group 200. In step S1104, the event determination section 112 determines presence or absence of occurrence of an event using the data from the operating room apparatus group 200, and outputs information regarding an occurrence zone of the event in a case of detecting occurrence of the event. In step S1108, the event zone analysis section 113 performs multiple regression analysis or the like on the data acquired in the event occurrence zone to thereby extract a factor that influences the data evaluating the surgical achievement. In step S1112, the event zone analysis section 113 determines statistical significance of the analysis result. In a case where the analysis result is determined to be statistically insignificant and where iteration number is equal to or less than a predetermined value (step S1116/No), a series of processing from step S1104 to step S1112 is performed again. In a case where the analysis result is determined to be statistically significant, or in a case where the analysis result is determined to be statistically insignificant and where the iteration number is larger than the predetermined value (step S1116/Yes), a series of processing ends.

It is to be noted that the steps in the flowcharts illustrated in FIGS. 8 and 9 need not necessarily be processed in time series in the described order. That is, the steps in the flowchart either may be processed in an order different from the described order, or may be processed in parallel.

### 1.5. Processing Variation

The description has been given above of an example of the flow of the processing by the medical information processor 100 according to the present embodiment. Subsequently, description is given of variations of the processing by the medical information processor 100 described above.

The description has been given above to the effect that the event determination section 112 appropriately changes the threshold value to be used in the processing of determining presence or absence of occurrence of an event to thereby be able to extract a more adequate factor. Here, the event determination section 112 may change a sampling interval (or sampling frequency) of the acquired data, instead of the threshold value, to thereby be able to extract a more adequate factor.

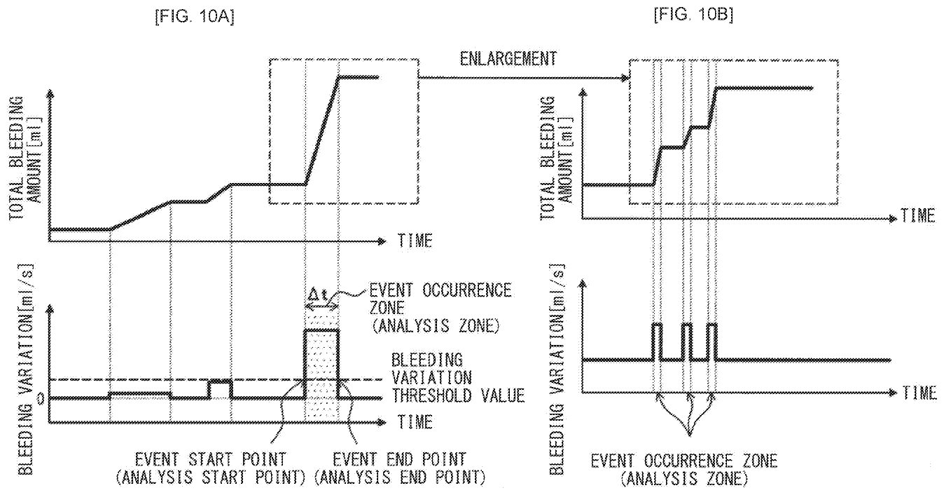

More specifically, in a case where the event zone analysis section 113 fails to extract any statistically significant factor, the event determination section 112 changes the sampling interval of the acquired data to be shorter (or changes the sampling frequency to be higher) as illustrated in the change from FIG. 10A to 10B. This allows the event determination section 112 to recognize the total bleeding amount and the bleeding variation more finely than FIG. 10A, thus making it possible to output the event occurrence zone more finely. For example, as illustrated in FIG. 10B, the event determination section 112 may be able to extract more event occurrence zones than the case before the change in the sampling interval. Accordingly, the event zone analysis section 113 may be more likely to extract a statistically significant factor. It is to be noted that a case may be assumed where too short sample interval may result in failure to extract a statistically significant factor. Therefore, the event determination section 112 may attempt a more accurate output by setting several types of sampling intervals and extracting event occurrence zones at respective sampling intervals.

In addition, as described above, the medical information processor 100 performs a feedback mainly during the surgery to improve the ongoing surgery. This is not limitative; the medical information processor 100 may perform a feedback to improve subsequent surgeries.

More specifically, as illustrated in FIGS. 11A and 11B, the event zone analysis section 113 sets, as an analysis zone, a zone before occurrence of an event (for example, as illustrated in FIGS. 11A and 11B, a zone from a start point of an event one period before to a start point of the event), and analyzes an indication of occurrence of the event. For example, the event zone analysis section 113 performs multiple regression analysis or the like on various data in the analysis zone before the occurrence of an event to thereby output the most suitable factor as the indication of the occurrence of an event. This enables the event zone analysis section 113 to perform a feedback to prevent occurrence of an event during a subsequent surgery. It is to be noted that, also in this processing, the threshold value to be used in the processing of determining presence or absence of occurrence of an event, and the sampling interval (or sampling frequency) of acquired data may be changed as appropriate.

### 2. SECOND EMBODIMENT

The description has been given above of the first embodiment according to the present disclosure. Subsequently, description is given of a second embodiment according to the present disclosure. The description has been given, in the above-described embodiment, of the example of a case where the medical information processor 100 performs a feedback on the basis of a correlation between the bleeding amount and the image quality parameter. Subsequently, description is given, as a second embodiment, of an example of a case where the medical information processor 100 performs a feedback on the basis of a correlation among BMI as presurgical information, a maker of an endoscopic system used in surgery as intra-surgical information, and the number of days of hospitalization as postsurgical information.

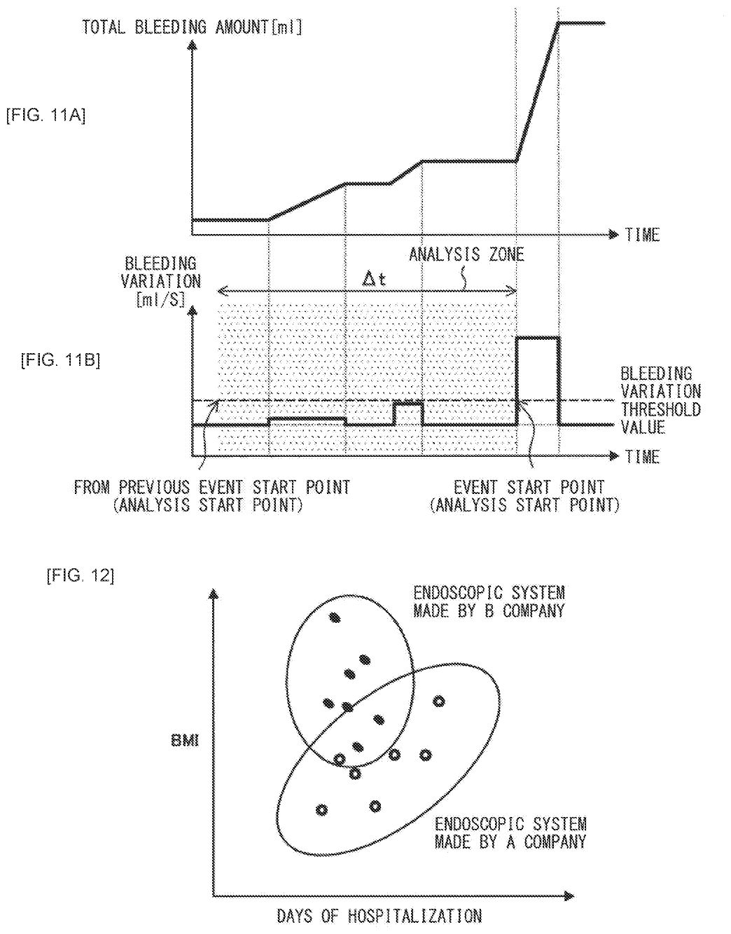

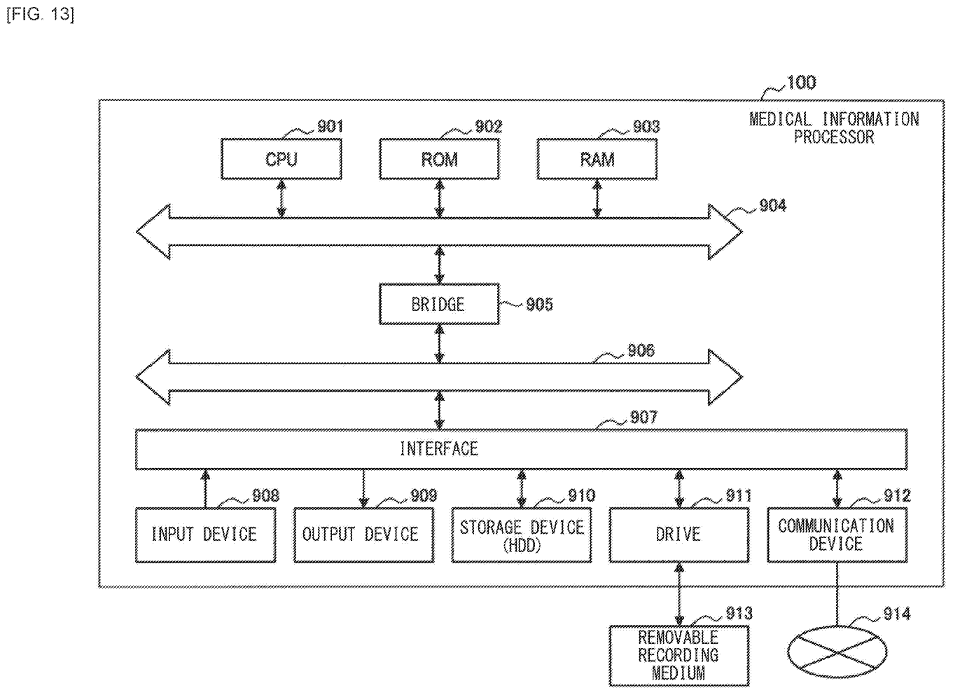

FIG. 12 illustrates a relationship between BMI and the number of days of hospitalization of a patient in each of a surgery using an endoscopic system made by A company or a surgery using an endoscopic system made by B company. It is to be noted that each plot in FIG. 12 indicates data regarding one surgery. As illustrated in FIG. 12, in a case where the endoscopic system made by the A company is used, positive correlations are confirmed between the BMI and the numbers of days of hospitalization of the patients. Meanwhile, in a case where the endoscopic system made by the B company is used, no correlations are confirmed between the BMI and the numbers of days of hospitalization of the patients, and the numbers of days of hospitalization are substantially constant (or the numbers of days of hospitalization fall within a certain range) regardless of the BMI.

The analysis unit 110 of the medical information processor 100 analyzes information acquired from the operating room apparatus group 200 and the patient data server 300 to thereby recognize this feature. Then, the analysis unit 110 performs a feedback to propose a maker of the endoscopic system to be used for the surgery on the basis of the BMI of a patient to be subjected to a new surgery. It is considered that this enables the operator to adequately determine the maker of the endoscopic system to be used for surgery at the stage of planning or preparation for the surgery, and thus to shorten the number of days of hospitalization of the patient. It is to be noted that those described above are merely exemplary, and the contents of the second embodiment may be changed as appropriate. For example, information from which correlations are extracted is not limited to the BMI, the endoscopic system maker, and the number of days of hospitalization.

### 3. THIRD EMBODIMENT

Subsequently, description is given of a third embodiment according to the present disclosure. In the foregoing embodiment, the description has been given of the case where the medical information processor 100, the operating room apparatus group 200, and the patient data server 300 are installed in the same hospital. In the third embodiment, a case is considered where the medical information processor 100 is implemented as a cloud server located on a cloud network, and the operating room apparatus group 200 and the patient data server 300 are installed in a plurality of hospitals.

The medical information processor 100 implemented as a cloud server is able to acquire data regarding more surgeries from a plurality of hospitals. Therefore, the medical information processor 100 is able to enhance a data amount to be used for analysis processing of the event occurrence zone, thus making it possible to improve accuracy in the analysis processing. In addition, a plurality of hospitals is able to receive feedback from the analysis processing of the medical information processor 100. It is to be noted that a specific implementing method or the like is not particularly limited. For example, not only the medical information processor 100 implemented as a cloud server is used, but also the medical information processor 100 may be installed in each of the hospitals to thereby allow for implementation of these medical information processors 100 to share the processing.

### 4. FOURTH EMBODIMENT

Subsequently, description is given of a fourth embodiment according to the present disclosure. The point of the present disclosure is to extract an unfounded correlation between data, as described above. In a case where a new apparatus (e.g., a product of another company or a new product, etc.), of which specifications (e.g., data unit, data format, data type (e.g., data dimension, etc.), delay information, apparatus type or various parameters (e.g., resolution or image quality parameter, etc.)) are unclear, is coupled to an operating room network system, it is usually required that the medical information processor 100 be able to process data outputted from the new apparatus by modifying the new apparatus or the medical information processor 100. However, this modification imposes a large load.

Therefore, in the present embodiment, in a case where a new apparatus is coupled as the operating room apparatus group 200 or the patient data server 300 to the operating room network system, an apparatus (hereinafter, for the sake of convenience, referred to as an “analysis apparatus”; the analysis apparatus functions as a second analysis unit) that analyzes output data from the new apparatus is separately installed between the new apparatus and the medical information processor 100. For example, in a case where a new endoscopic system made by another company is coupled as the operating room apparatus group 200 to the operating room network system, an IP-converter that analyzes output data from the endoscopic system made by the other company may be installed between the new endoscopic system made by the other company and the medical information processor 100.

The analysis apparatus analyses (e.g., analyzes a frequency, etc.) pre-encoded baseband signals and the like outputted from the new apparatus coupled to the operating room network system to thereby recognize specifications (e.g., data unit, data format, data type (e.g., data dimension, etc.), delay information, apparatus type, or various parameters (e.g., resolution or image quality parameter, etc.)) of the new apparatus. Then, the analysis apparatus provides results of the analysis to the medical information processor 100, thereby enabling the medical information processor 100 to perform the analysis processing, the feedback, and the like described above using data outputted from the new apparatus, even without modification, or the like. It is to be noted that the analysis apparatus may perform not only the analysis of the data outputted from the new apparatus, but also processing, etc. of converting the data outputted from the new apparatus into data processable by the medical information processor 100.

### 5. OTHER EMBODIMENTS

The description has been given above of various embodiments according to the present disclosure. However, those described above are merely exemplary, and the embodiment to which the present disclosure is applied is not limited to those described above. Description is now given below of other embodiments to which the present disclosure is applied.

For example, the present disclosure may be applied to a case of using, as the operating room apparatus group 200, an energy device such as an electric scalpel and a smoke ventilation apparatus for discharging smoke generated by use of the energy device. To describe more specifically, application of the present disclosure allows the analysis unit 110 to determine presence or absence of occurrence of an event of smoke generation and to analyze various data (e.g., energization pattern of the energy device or operation status of the smoke ventilation apparatus (e.g., smoke ventilation status, etc.)) in a zone in which the event has occurred, to thereby be able to compare performance of the smoke ventilation apparatus used during the surgery and that of another smoke ventilation apparatus with each other. This enables the analysis unit 110 to select a smoke ventilation apparatus that is able to perform smoke ventilation more quickly and to perform a feedback during the surgery in a case where the surgery time is required to be reduced.

In addition, the present disclosure may be applied to a case of using, as the operating room apparatus group 200, an energy device such as an electric scalpel and a surgical field camera that captures an image of a blood tank (or a blood tank itself). More specifically, the analysis unit 110 analyzes a captured image of the surgical field camera to thereby determine presence or absence of occurrence of an event of bleeding, and analyzes various data (e.g., energization pattern of the energy device or accumulation amount of blood in the blood tank) in the zone in which the event has occurred to thereby be able to compare performance of the energy device used during the surgery and that of another energy device with each other. This enables the analysis unit 110 to select an energy device that is able to suppress the bleeding amount and to perform a feedback during the surgery in a case where the bleeding amount is required to be suppressed (or the bleeding time is required to be shortened).

In the examples described above in which the energy device and the smoke ventilation apparatus are used and in which the energy device and the surgical field camera (or the blood tank itself) are used, the present disclosure is particularly effective in a case where the apparatus is susceptible to aged deterioration (or the apparatus is susceptible to failure), in a case where there is an individual difference in the performances of the apparatuses, or the like. For example, the performance of the smoke ventilation apparatus varies greatly due to aged deterioration, etc., and thus it may be difficult, in some cases, to predict the performance of the smoke ventilation apparatus from specifications disclosed in a catalog. Accordingly, the operator actually uses the smoke ventilation apparatus to thereby confirm the performance thereof. From those described above, it is difficult to quantitatively measure the performance of the smoke ventilation apparatus and to make a comparison with another smoke ventilation apparatus; however, the present disclosure is able to select a more adequate smoke ventilation apparatus and to perform a feedback during the surgery, and thus is particularly effective.

### 6. HARDWARE CONFIGURATION EXAMPLE

The description has been given above of various embodiments according to the present disclosure. Subsequently, description is given, with reference to FIG. 13, of a hardware configuration example of the medical information processor 100.

FIG. 13 is a block diagram illustrating the hardware configuration example of the medical information processor 100. The medical information processor 100 includes a CPU (Central Processing Unit) 901, a ROM (Read Only Memory) 902, a RAM (Random Access Memory) 903, a host bus 904, a bridge 905, an external bus 906, an interface 907, an input device 908, an output device 909, a storage device (HDD) 910, a drive 911, and a communication device 912.

The CPU 901 functions as an arithmetic processing device and a control device, and controls overall operations in the medical information processor 100 in accordance with various programs. In addition, the CPU 901 may be a microprocessor. The ROM 902 stores programs to be used by the CPU 901, arithmetic parameters, and the like. The RAM 903 temporarily stores programs to be used in execution by the CPU 901, parameters that vary appropriately in executing the program, and the like. These components are coupled mutually by the host bus 904 configured by a CPU bus, or the like. The cooperation among the CPU 901, the ROM 902 and the RAM 903 implements the functions of the analysis unit 110 of the medical information processor 100.

The host bus 904 is coupled through the bridge 905 to the external bus 906 such as a PCI (Peripheral Component Interconnect/Interface) bus. It is to be noted that the host bus 904, the bridge 905, and the external bus 906 need not necessarily be configured separately, and these functions may be implemented in one bus.

The input device 908 is configured by input means for a user to input information such as a mouse, a keyboard, a touch panel, a button, a microphone, a switch and a lever, and an input control circuit to generate an input signal on the basis of an input by a user and to output the generated input signal to the CPU 901. For example, the user using the medical information processor 100 manipulates the input device 908 to thereby be able to input various data to the medical information processor 100 or instruct the medical information processor 100 to perform a processing operation.

The output device 909 includes, for example, a display device such as a CRT (Cathode Ray Tube) display device, a liquid crystal display (LCD) device, an OLED (Organic Light Emitting Diode) device, and a lamp. Further, the output device 909 includes a sound output device such as a speaker and a headphone. The output device 909 outputs a reproduced content, for example. Specifically, the display device displays various types of information such as reproduced image data, in the form of a character string or an image. Meanwhile, the sound output device converts reproduced sound data or the like into a sound to output the converted sound.

The storage device 910 is a device for storing data. The storage device 910 may include a storage medium, a recording device that records data on the storage medium, a reading device that reads data from the storage medium, and a deleting device that deletes data recorded on the storage medium. The storage device 910 is configured by, for example, an HDD (Hard Disk Drive). The storage device 910 drives the hard disk, and stores programs to be executed by the CPU 901 and various data. The storage device 910 implements the functions of the storage unit 130 of the medical information processor 100.

The drive 911 is a reader/writer for a storage medium, and is built in or externally attached to the medical information processor 100. The drive 911 reads information recorded in a removable recording medium 913 such as a magnetic disk, an optical disk, a magneto-optical disk, or a semiconductor memory mounted thereon, and outputs the read information to the RAM 903. In addition, the drive 911 is also able to write information into the removable recording medium 913.

The communication device 912 is a communication interface configured by, for example, a communication device for being coupled to a communication network 914. The communication device 912 implements functions of the communication unit 120 of the medical information processor 100.

### 7. CONCLUSION

As described above, the medical information processor 100 according to the present disclosure acquires output data of a plurality of apparatuses coupled to the operating room network (e.g., a plurality of apparatuses included in the operating room apparatus group 200) and analyzes the output data to thereby be able to adequately perform a feedback. Accordingly, unlike the apparatus disclosed in PTL 1, etc., it is possible for the medical information processor 100 to sufficiently utilize output data from a plurality of apparatuses used during surgery.

In addition, the medical information processor 100 is able to calculate, with respect to output data acquired from a plurality of apparatuses coupled to the operating room network, a correlation between data evaluating a surgical achievement and other output data, and is able to perform an adequate feedback on the basis of the correlation. That is, the medical information processor 100 has an advantage distinctly different from the apparatus disclosed in PTL 1, etc. which performs a feedback from data which has been found to be correlated in advance.

Further, the medical information processor 100 analyzes the output data acquired during surgery to thereby be able to perform an adequate feedback during the surgery. This enables the medical information processor 100 to improve surgical procedures in real time and thus to reduce surgical failures. It is to be noted that, as described above, the medical information processor 100 may analyze output data acquired during time outside surgery (e.g., before or after the surgery) and may perform a feedback during the time outside the surgery (e.g., before or after the surgery).

Although the description has been given above in detail of preferred embodiments of the present disclosure with reference to the accompanying drawings, the technical scope of the present disclosure is not limited to such examples. It is obvious that a person having ordinary skill in the art of the present disclosure may find various alterations or modifications within the scope of the technical idea described in the claims, and it should be understood that these alterations and modifications naturally come under the technical scope of the present disclosure.

In addition, the effects described herein are merely illustrative or exemplary, and are not limitative. That is, the technology according to the present disclosure may achieve, in addition to or in place of the above effects, other effects that are obvious to those skilled in the art from the description of the present specification.

It is to be noted that the technical scope of the present disclosure also includes the following configurations.

(1)

A medical information processor including:

an acquisition unit that acquires output data of a plurality of apparatuses coupled to an operating room network;

a first analysis unit that performs an analysis on a basis of a correlation among the output data; and

an output control section that controls an output of a result of the analysis.

(2)

The medical information processor according to (1), in which the output data include data evaluating a surgical achievement.

(3)

The medical information processor according to (2), in which the data evaluating the surgical achievement include data regarding a bleeding amount, data regarding surgery time, data regarding a hospitalization period, or data regarding a survival rate or a complication occurrence rate.

(4)

The medical information processor according to (2) or (3), in which the first analysis unit detects an event being an event that influences the data evaluating the surgical achievement on a basis of the output data.

(5)

The medical information processor according to (4), in which the first analysis unit specifies a period during which the event has occurred on a basis of a temporal change of the output data.

(6)

The medical information processor according to (5), in which the first analysis unit extracts a correlation between the data evaluating the surgical achievement and the output data in the period.

(7)

The medical information processor according to any one of (4) to (6), in which the first analysis unit changes a threshold value or a sampling interval of the output data used to detect the event in a case where the result of the analysis is not statistically significant.

(8)

The medical information processor according to any one of (1) to (7), in which the output data are acquired from a plurality of hospitals.

(9)

The medical information processor according to any one of (1) to (8), in which
the acquisition unit acquires the output data during surgery,
   the first analysis unit performs the analysis during the surgery, and
   the output control section controls the output during the surgery.

(10)

The medical information processor according to any one of (1) to (9), in which the output control section controls an output of information regarding a method for improving the surgical achievement as the result of the analysis.

(11)

The medical information processor according to (10), in which the output control section controls a display content, a display size, a display color, a display position, a content of a sound output, a magnitude of the sound output, lighting or flashing of a lamp in the output, in accordance with a degree of recommendation or reliability of the information regarding the method for improving the surgical achievement.

(12)

The medical information processor according to any one of (1) to (11), further including a second analysis unit that analyzes output data of an apparatus of which a specification is unclear, and provides a result of the analysis to the first analysis unit.

(13) A medical information processing method implemented by a computer, the method including:
acquiring output data of a plurality of apparatuses coupled to an operating room network;
performing an analysis on a basis of a correlation among the output data; and
controlling an output of a result of the analysis.

An operating room network system including:
a plurality of apparatuses coupled to an operating room network; and
a medical information processor that analyzes output data of the plurality of apparatuses, the medical information processor including
   an acquisition unit that acquires the output data,
a first analysis unit that performs an analysis on a basis of a correlation among the output data, and
an output control section that controls an output of a result of the analysis.

### REFERENCE NUMERALS LIST

100 medical information processor
110 analysis unit
111 delay adjustment section
112 event determination section
113 event zone analysis section
114 output control section
120 communication unit
130 storage unit
200 operating room apparatus group
300 patient data server
400a, 400b network

1. A medical information processor comprising:
an acquisition unit that acquires output data of a plurality of apparatuses coupled to an operating room network;
a first analysis unit that performs an analysis on a basis of a correlation among the output data; and
an output control section that controls an output of a result of the analysis.

2. The medical information processor according to claim 1, wherein the output data include data evaluating a surgical achievement.

3. The medical information processor according to claim 2, wherein the data evaluating the surgical achievement include data regarding a bleeding amount, data regarding surgery time, data regarding a hospitalization period, or data regarding a survival rate or a complication occurrence rate.

4. The medical information processor according to claim 2, wherein the first analysis unit detects an event being an event that influences the data evaluating the surgical achievement on a basis of the output data.

5. The medical information processor according to claim 4, wherein the first analysis unit specifies a period during which the event has occurred on a basis of a temporal change of the output data.

6. The medical information processor according to claim 5, wherein the first analysis unit extracts a correlation between the data evaluating the surgical achievement and the output data in the period.

7. The medical information processor according to claim 4, wherein the first analysis unit changes a threshold value or a sampling interval of the output data used to detect the event in a case where the result of the analysis is not statistically significant.

8. The medical information processor according to claim 1, wherein the output data are acquired from a plurality of hospitals.

9. The medical information processor according to claim 1, wherein
the acquisition unit acquires the output data during surgery,
the first analysis unit performs the analysis during the surgery, and
the output control section controls the output during the surgery.

10. The medical information processor according to claim 1, wherein the output control section controls an output of information regarding a method for improving a surgical achievement as the result of the analysis.

11. The medical information processor according to claim 10, wherein the output control section controls a display content, a display size, a display color, a display position, a content of a sound output, a magnitude of the sound output, lighting or flashing of a lamp in the output, in accordance with a degree of recommendation or reliability of the information regarding the method for improving the surgical achievement.

12. The medical information processor according to claim 1, further comprising a second analysis unit that analyzes output data of an apparatus of which a specification is unclear, and provides a result of the analysis to the first analysis unit.

13. A medical information processing method implemented by a computer, the method comprising:
acquiring output data of a plurality of apparatuses coupled to an operating room network;
performing an analysis on a basis of a correlation among the output data; and
controlling an output of a result of the analysis.

14. An operating room network system comprising:
a plurality of apparatuses coupled to an operating room network; and
a medical information processor that analyzes output data of the plurality of apparatuses, the medical information processor including
an acquisition unit that acquires the output data,
a first analysis unit that performs an analysis on a basis of a correlation among the output data, and
an output control section that controls an output of a result of the analysis.
